(19) United States
(12) Patent Application Publication
(10) Pub. No.: US 2025/0171718 A1
(43) Pub. Date: May 29, 2025

(54) SEMICONDUCTOR CLEANING LIQUID AND METHOD FOR PRODUCING SEMICONDUCTOR CLEANING LIQUID

(71) Applicant: TOKUYAMA CORPORATION, Yamaguchi (JP)

(72) Inventors: Shunsuke Hosaka, Yamaguchi (JP); Takashi Tokunaga, Yamaguchi (JP); Yoshiaki Yamashita, Yamaguchi (JP)

(73) Assignee: TOKUYAMA CORPORATION, Yamaguchi (JP)

(21) Appl. No.: 18/842,549

(22) PCT Filed: Feb. 7, 2023
(86) PCT No.: PCT/JP2023/003901

§ 371 Date: Aug. 29, 2024

(30) Foreign Application Priority Data
Mar. 16, 2022 (JP) 2022-041318

(51) Int. Cl. C11D 7/26; C07C 29/04; C07C 29/80

(52) U.S. Cl. CPC C11D 7/261; C07C 29/04; C07C 29/80; C11D 7/264; C11D 2111/22

(57) ABSTRACT

The present invention provides a semiconductor cleaning liquid which contains isopropyl alcohol, while having a mass ratio of t-butyl alcohol of 1 ppm or less relative to the isopropyl alcohol. The present invention also provides a method for producing a semiconductor cleaning liquid, the method comprising a first distillation step in which a crude aqueous isopropyl alcohol solution that contains t-butyl alcohol as an impurity is supplied to a starting material supply plate of a first distillation column, and a first liquid distillate which contains a low boiling point impurity that has a lower boiling point than isopropyl alcohol is extracted from the column top of the first distillation column, while extracting a first bottom liquid from the column bottom of the first distillation column. In the first distillation step, a fluid containing water is supplied, from the outside of the first distillation column, to a predetermined plate that is above the starting material supply plate by two or more theoretical plates so that the water content in the liquid phase is 15% by mass or more at three or more theoretical plates among the plates of the first distillation column from the starting material supply plate to the column top.

### TECHNICAL FIELD

The present invention relates to a semiconductor cleaning liquid and a method for producing a semiconductor cleaning liquid.

### BACKGROUND ART

Conventionally, in a semiconductor manufacturing process, a substrate such as a semiconductor substrate or a glass substrate is cleaned with a semiconductor cleaning liquid and then dried. As the semiconductor cleaning liquid, for example, isopropyl alcohol is used.

As a method for producing isopropyl alcohol, for example, a direct hydration method of propylene (see Patent Document 1) is known.

### CITATION LIST

### Patent Document

Patent Document 1: PCT International Publication No. WO2017/217279

### DISCLOSURE OF THE INVENTION

### Problems to be Solved by the Invention

However, since isopropyl alcohol produced by the direct hydration method of propylene contains t-butyl alcohol as an impurity, there is a concern that a residue of t-butyl alcohol adversely affects a semiconductor device when such isopropyl is used as a semiconductor cleaning liquid. Therefore, although reducing a content of t-butyl alcohol in the semiconductor cleaning liquid is desired, it is difficult to separate t-butyl alcohol because t-butyl alcohol has substantially the same boiling point, 82° C., as isopropyl alcohol.

An object of the present invention is to provide a semiconductor cleaning liquid having a reduced content of t-butyl alcohol and a method for producing a semiconductor cleaning liquid, the method being capable of reducing a content of t-butyl alcohol.

### Means for Solving the Problems

An aspect of the present invention is a semiconductor cleaning liquid containing isopropyl alcohol, in which a mass ratio of t-butyl alcohol with respect to isopropyl alcohol is 1 ppm or less.

In the semiconductor cleaning liquid, a mass ratio of pentanone with respect to isopropyl alcohol may be 1 ppb or more and 50 ppb or less, and a mass ratio of crotonaldehyde with respect to isopropyl alcohol may be 0.5 ppb or more and 10 ppb or less.

Isopropyl alcohol may be prepared by direct hydration of propylene.

Another aspect of the present invention is a method for producing a semiconductor cleaning liquid, the method including a first distillation step of supplying a crude isopropyl alcohol aqueous solution containing t-butyl alcohol as an impurity to a feedstock supply plate of a first distillation column, withdrawing a first distillate containing a low-boiling point impurity having a lower boiling point than isopropyl alcohol from a column top of the first distillation column, and withdrawing a first bottom liquid from a column bottom of the first distillation column. In the first distillation step, a fluid containing water is supplied from outside the first distillation column to a predetermined plate which is two or more theoretical plates above the feedstock supply plate of the first distillation column so that a water content in a liquid phase is 15 mass % or more in three or more, as a theoretical plate number, plates among plates of the first distillation column from the feedstock supply plate to the column top.

The method for producing a semiconductor cleaning liquid may further include a second distillation step of supplying the first distillate to a feedstock supply plate of a second distillation column, withdrawing a second distillate containing the low-boiling point impurity from the column top of the second distillation column, and withdrawing a second bottom liquid from the column bottom of the second distillation column. In the second distillation step, water is supplied to a predetermined plate of the second distillation column from outside the second distillation column, and in the first distillation step, the second bottom liquid may be supplied as the liquid containing water.

The predetermined plate of the second distillation column may be a plate between the feedstock supply plate of the second distillation column and the column top thereof.

The method for producing a semiconductor cleaning liquid may further include a reaction step of obtaining the crude isopropyl alcohol aqueous solution by a direct hydration method of propylene.

### Effects of the Invention

According to the present invention, it is possible to provide a semiconductor cleaning liquid having a reduced content of t-butyl alcohol and a method for producing a semiconductor cleaning liquid capable of reducing the content of t-butyl alcohol.

### BRIEF DESCRIPTION OF THE DRAWINGS

FIG. 1 is an x-y diagram of t-butyl alcohol/isopropyl alcohol when a water content is 08 by mass;

FIG. 2 is an x-y diagram of t-butyl alcohol/isopropyl alcohol when the water content is 18% by mass;

FIG. 3 is an x-y diagram of t-butyl alcohol/isopropyl alcohol when the water content is 36% by mass;

FIG. 4 is an x-y diagram of t-butyl alcohol/isopropyl alcohol when the water content is 54 mass %;

FIG. 5 is an x-y diagram of t-butyl alcohol/isopropyl alcohol when the water content is 72% by mass;

FIG. 6 is an x-y diagram of t-butyl alcohol/isopropyl alcohol when the water content is 90% by mass;

FIG. 7 is an x-y diagram of water/isopropyl alcohol;

FIG. 8 is a diagram showing an example of a low-boiling distillation step in the method for producing a semiconductor cleaning liquid according to the present embodiment; and

FIG. 9 is a view showing another example of the low-boiling distillation step in the method for producing the semiconductor cleaning liquid of the present embodiment.

### PREFERRED MODE FOR CARRYING OUT THE INVENTION

Hereinafter, embodiments of the present invention will be described with reference to the drawings.

### <Semiconductor Cleaning Liquid>

The semiconductor cleaning liquid of the present embodiment contains isopropyl alcohol, and a mass ratio of t-butyl alcohol is 1 ppm or less, more preferably 0.5 ppm or less, and still more preferably 0.3 ppm or less with respect to isopropyl alcohol. Therefore, even when a substrate is cleaned using the semiconductor cleaning liquid of the present embodiment, residue of t-butyl alcohol does not adversely affect a semiconductor device. The mass ratio of t-butyl alcohol with respect to isopropyl alcohol is not particularly limited, but is preferably 0.01 ppm or more, and more preferably 0.1 ppm or more, because the cost increases when the mass ratio is too low. The mass ratio of t-butyl alcohol with respect to isopropyl alcohol is measured by gas chromatography mass spectrometry (GC-MS). When a water concentration of the semiconductor cleaning liquid is less than 18, the measurement may be performed by directly introducing the semiconductor cleaning liquid into an injection of an apparatus, but when the water concentration of the semiconductor cleaning liquid is 1% or more, the measurement is preferably performed by a headspace-GC-MS method.

Isopropyl alcohol can be produced by, for example, a direct hydration method of propylene.

In this case, even if the feedstock propylene does not contain isobutene as an impurity, since t-butyl alcohol is produced as a by-product, it is presumed that isobutene is produced by disproportionation reaction of propylene during production of isopropyl alcohol (see the following reaction mechanism).

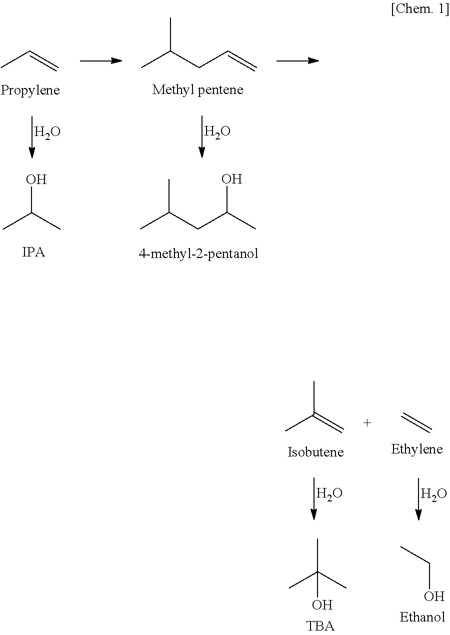

Isopropyl alcohol produced by the direct hydration method of propylene inevitably contains 2-pentanone, crotonaldehyde, and the like as impurities. A mass ratio of 2-pentanone with respect to isopropyl alcohol in the semiconductor cleaning liquid of the present embodiment is not particularly limited, but is, for example, 1 ppb or more and 50 ppb or less, and preferably 2 ppb or more and 30 ppb or less. A mass ratio of crotonaldehyde with respect to isopropyl alcohol in the semiconductor cleaning liquid of the present embodiment is not particularly limited, but is, for example, 0.5 ppb or more and 20 ppb or less, and preferably 2 ppb or more and 10 ppb or less.

A content of isopropyl alcohol in the semiconductor cleaning liquid of the present embodiment is preferably 99.99% by mass or more, and more preferably 99.999% by mass or more as expressed in a content excluding water.

The water content in the semiconductor cleaning liquid of the present embodiment is not particularly limited, but is, for example, 0.1 ppm by mass or more and 100 ppm by mass or less, and preferably 1 ppm or more and 20 ppm or less.

The semiconductor cleaning liquid of the present embodiment can be produced by a method for producing the semiconductor cleaning liquid of the present embodiment described later.

<Method for producing Semiconductor Cleaning Liquid>

A method for producing a semiconductor cleaning liquid of the present embodiment includes a first distillation step of supplying a crude isopropyl alcohol aqueous solution containing t-butyl alcohol as an impurity to a feedstock supply plate of a first distillation column, withdrawing a first distillate containing a low-boiling point impurity having a lower boiling point than isopropyl alcohol from the column top of the first distillation column, and withdrawing a first bottom liquid from the column bottom of the first distillation column. At this time, in the first distillation step, a fluid containing water is supplied from outside the first distillation column to a predetermined plate which is two or more theoretical plates above, more preferably three or more theoretical plates above, and still more preferably five or more theoretical plates above, the feedstock supply plate of the first distillation column so that a water content in a liquid phase is 15 mass % or more in three or more, as a theoretical plate number, plates among the plates of the first distillation column from the feedstock supply plate to the column top. This reduces the content of t-butyl alcohol in the semiconductor cleaning liquid. At this time, the water content in the liquid phase is preferably 15% by mass or more in three or more, as a theoretical plate number, plates, and more preferably four or more plates, among the plates of the first distillation column from the feedstock supply plate to the column top thereof.

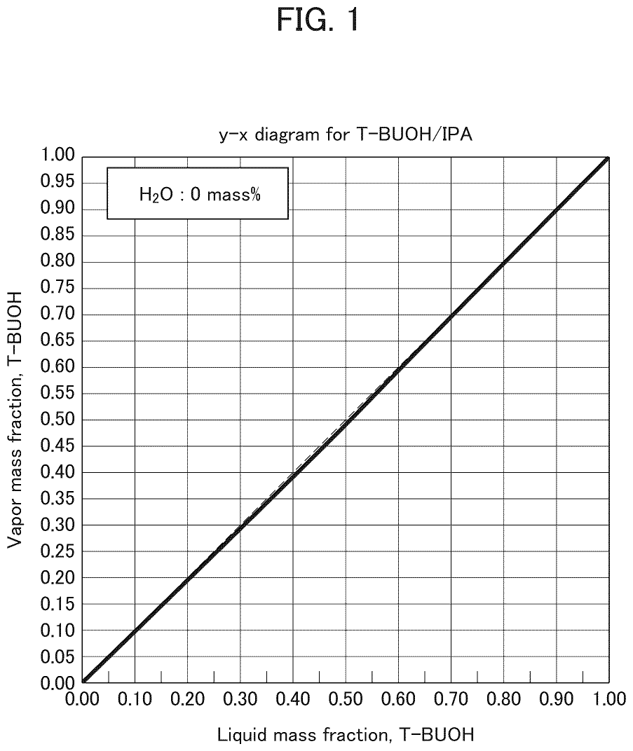

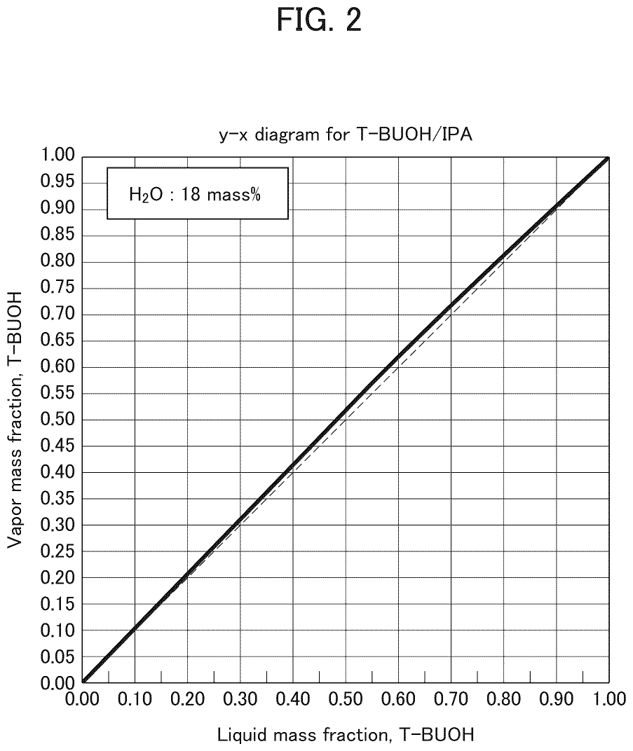

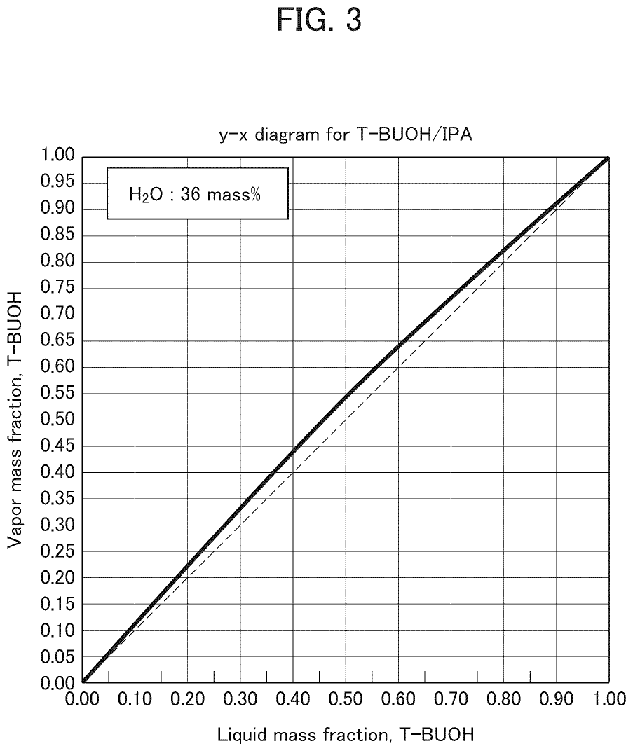

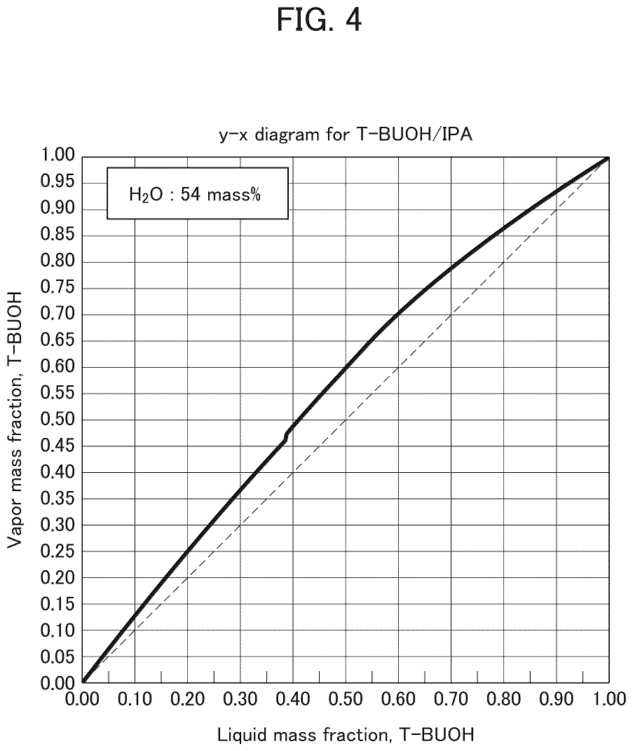

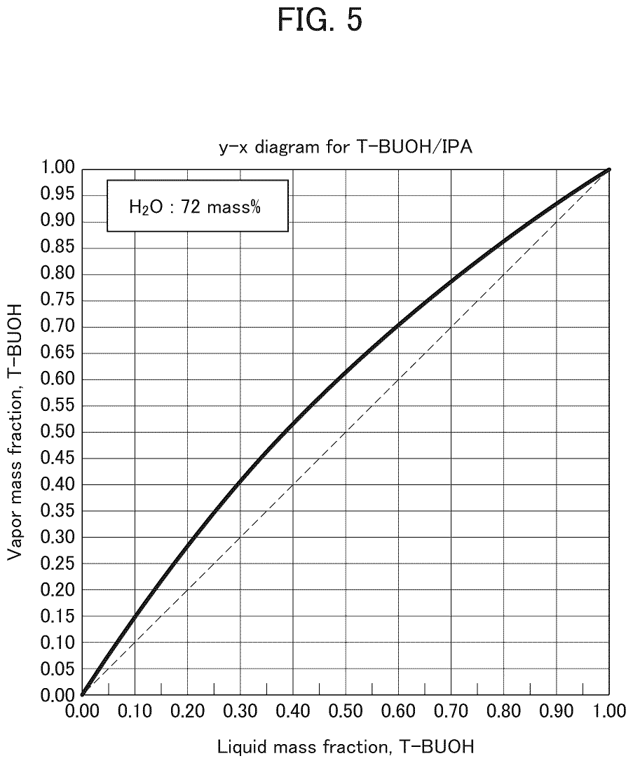

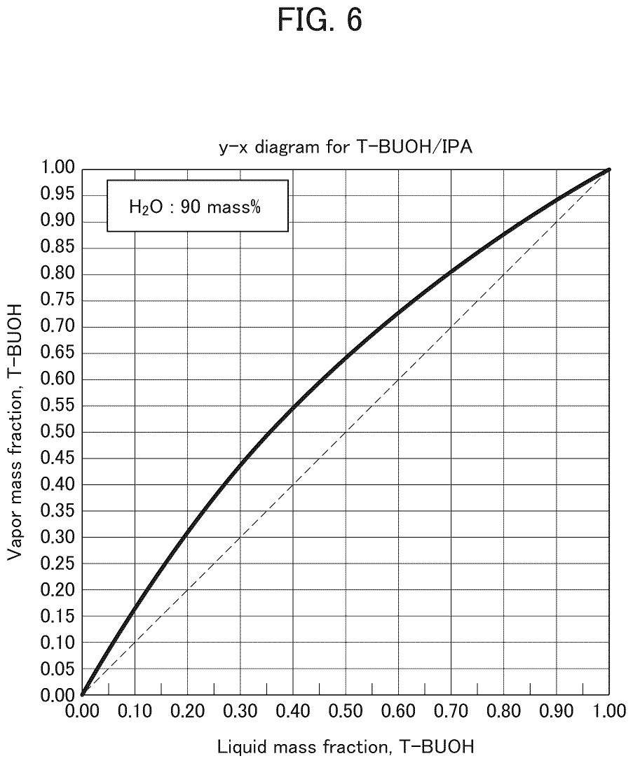

FIGS. 1 to 6 show x-y diagrams of t-butyl alcohol/isopropyl alcohol when the water content is 0 to 90% by mass.

It can be seen from FIGS. 1 to 6 that separation of isopropyl alcohol and t-butyl alcohol is promoted in a case where the water content is 18 to 90% by mass as compared with a case where the water content is 0% by mass. Therefore, the content of t-butyl alcohol in the semiconductor cleaning liquid is reduced by setting the water content in the liquid phases to 15 mass % or more in three or more, as a theoretical plate number, plates among the plates of the first distillation column from the feedstock supply plate to the column top.

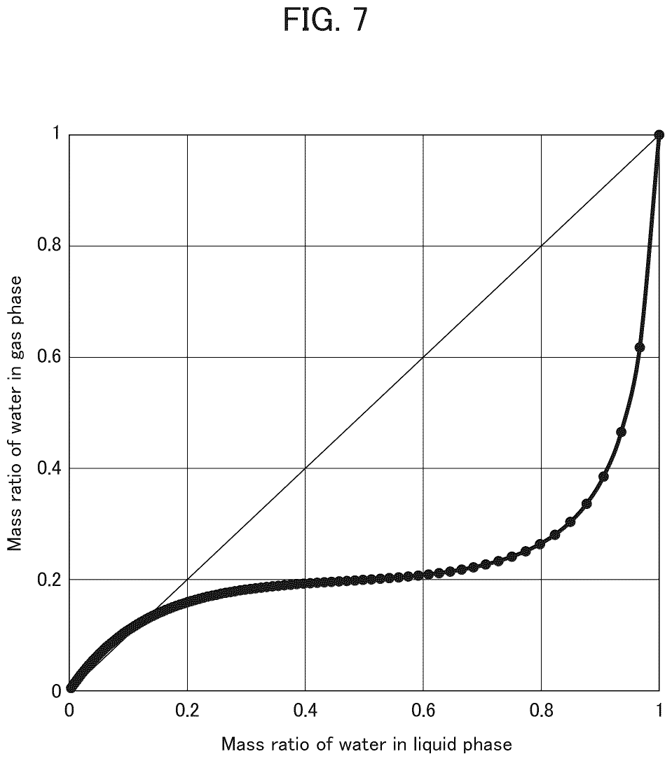

FIG. 7 shows an x-y diagram of water/isopropyl alcohol.

It can be seen from FIG. 7 that, for example, a water/isopropyl alcohol mixture having a water content of 95 mass % comes to have a water content of 20 mass % in two theoretical plates; a water content of 15 mass % in four theoretical plates; and a water content of 13 mass % (a water content of 12.5 mass % in an azeotropic composition) in six theoretical plates. Therefore, the fluid containing water can be supplied from outside the first distillation column to a predetermined plate which is two or more theoretical plates above the feedstock supply plate of the first distillation column so that the water content in the liquid phase is 15 mass % or more in three or more, as a theoretical plate number, plates among the plates of the first distillation column from the feedstock supply plate to the column top.

The fluid containing water may be a gas or a liquid. The water content in the fluid containing water and the amount of water to be supplied may be appropriately adjusted. The water content in the fluid containing water is preferably 50% by mass or more, and more preferably 80% by mass or more. A mass ratio of an amount of water supplied with respect to an amount of the feedstock (crude isopropyl alcohol aqueous solution containing t-butyl alcohol as an impurity) supplied is preferably 1/10 to 1/1,000, and more preferably 1/50 to 1/200. When the amount of water supplied is too large, an amount of water in the system becomes large, so that it is necessary to increase a column diameter of the distillation column and it is necessary to take out excess water from the system of the entire process. When the amount of water supplied is too small, separation of t-butyl alcohol becomes insufficient.

In a preferred embodiment, water removed in an azeotropic distillation step and a dehydration step to be described later can be used in a reaction step to be described later, and water supplied to the first distillation column can be used as water to be used in the reaction step and additional water for compensating for slight water loss during operation.

### [Reaction Step]

The method for producing the semiconductor cleaning liquid of the present embodiment may further include a reaction step of obtaining a crude isopropyl alcohol aqueous solution by a direct hydration method of propylene. The reaction of propylene in the reaction step is represented by the following formula:

C₃H₆+H₂O→CH₃CH(OH)CH₃

By carrying out such a reaction in a reaction column, a reaction product containing isopropyl alcohol is obtained.

In the reaction step, temperature and pressure in the reaction column are preferably 200° C. or higher and 300° C. or lower and 150 atm or higher and 250 atm or lower, respectively. . . . In the reaction step, for example, various polyanion acid catalysts such as molybdenum-based and tungsten-based inorganic ion exchangers can be used. As the acid catalyst, phosphotungstic acid, silicotungstic acid and silicomolybdic acid are preferable from the viewpoint of reaction activity.

Reaction products dissolved in water are withdrawn from the reaction column. Then, by cooling the reaction products and reducing the pressure, unreacted propylene dissolved in water is recovered as a gas to obtain a crude isopropyl alcohol aqueous solution. The recovered propylene is reused as the feedstock. The water content in the crude isopropyl alcohol aqueous solution is preferably 80% by mass or more, and more preferably 90% by mass or more.

### (Low-Boiling Distillation Step)

The method for producing the semiconductor cleaning liquid of the present embodiment includes a first distillation step as a low-boiling distillation step of distilling the crude isopropyl alcohol aqueous solution obtained in the reaction step.

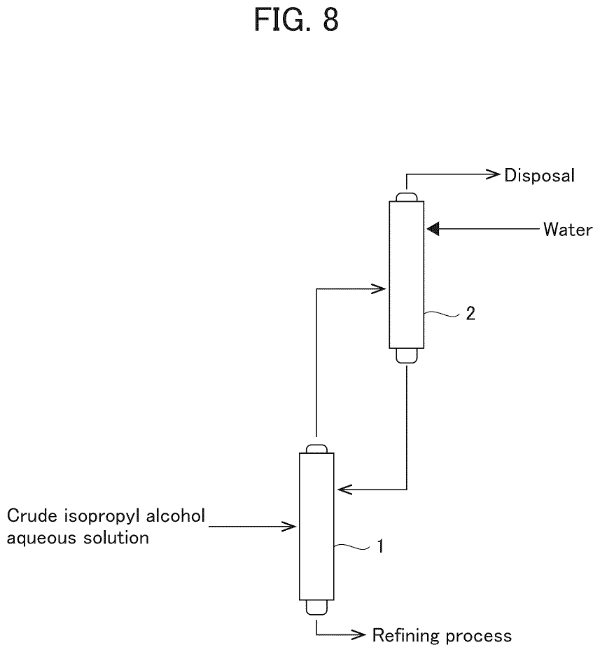

FIG. 8 shows an example of the low-boiling distillation step.

The crude aqueous isopropyl alcohol solution is supplied to the feedstock supply plate of the low-boiling distillation column (first distillation column) 1 via a conduit and distilled. At this time, a condenser is provided at the column top of the first distillation column 1, a part of liquid condensed by the condenser is refluxed, and the remainder is withdrawn as a first distillate. Further, the first bottom liquid is withdrawn from the column bottom of the first distillation column 1 and is purified.

Here, a reflux ratio of the first distillation column 1 is not particularly limited, but is, for example, 10 or more and 100 or less, and preferably 50 or more and 80 or less.

The first distillate withdrawn from the column top of the first distillation column 1 is supplied to the feedstock supply plate of a recovery distillation column (second distillation column) 2 And is distilled. At this time, a condenser is provided at the column top of the second distillation column 2, a part of the liquid condensed by the condenser is refluxed, and the remainder is withdrawn as a second distillate and is discharged. Further, while water is supplied to a predetermined plate of the second distillation column 2, water is preferably supplied to a plate between the feedstock supply plate of the second distillation column 2 and the column top thereof. Furthermore, it is preferable that the second bottom liquid is withdrawn from the column bottom of the second distillation column 2 and is supplied to a predetermined plate which is two or more theoretical plates above the feedstock supply plate of the first distillation column 1. It is more preferable that the second bottom liquid is supplied to a predetermined plate which is five or more theoretical plates above the feedstock supply plate of the first distillation column 1.

Here, a reflux ratio of the second distillation column 2 is not particularly limited, but is, for example, 5 or more and 50 or less, and preferably 10 or more and 30 or less.

Examples of low-boiling point impurities contained in the first distillate and the second distillate include olefins such as ethylene, propylene, butenes, pentenes, and hexenes; alkanes such as methane, ethane, propane, butane, pentane, and hexane; aldehydes such as acetaldehyde and propylenealdehyde; and ketones such as acetone and butanone.

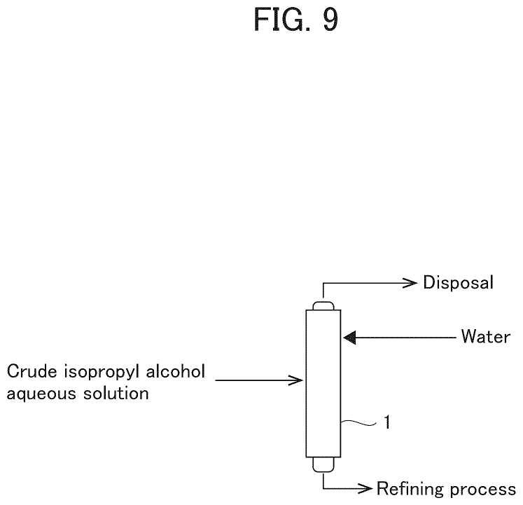

FIG. 9 shows another example of the low-boiling distillation step. FIG. 9 is in the same configuration as FIG. 8 except that the second distillation column 2 is omitted and water is supplied instead of the second bottom liquid to a predetermined plate which is two or more theoretical plates above the feedstock supply plate of the first distillation column 1.

The crude isopropyl alcohol aqueous solution is supplied to the feedstock supply plate of the low-boiling distillation column (first distillation column) 1 via a conduit and is distilled. At this time, a condenser is provided at the column top of the first distillation column 1, a part of the liquid condensed by the condenser is refluxed, and the remainder is withdrawn as a first distillate and is discharged. Further, water is supplied to a predetermined plate which is two or more plates above the feedstock supply plate of the first distillation column 1. Further, the first bottom liquid is withdrawn from the column bottom of the first distillation column 1 and is purified.

Here, a reflux ratio of the first distillation column 1 is not particularly limited, but is, for example, 10 or more and 100 or less, and preferably 50 or more and 80 or less.

Note that each of the first distillation column 1 and the second distillation column 2 may be either a plate column or a packed column, but is preferably a plate column. The number of theoretical plates of each distillation column is not particularly limited, but the number of theoretical plates of the first distillation column 1 is preferably 5 to 80 plates, and more preferably 10 to 50 plates. The number of theoretical plates of the second distillation column 2 is preferably 3 to 20, and more preferably 5 to 15. The number of actual plates in a case where the first distillation column 1 is a plate column may be appropriately adjusted so as to be the number of theoretical plates described above, and is, for example, 10 plates or more and 100 plates or less, and preferably 20 plates or more and 70 plates or less. When the second distillation column 2 is a plate column, the number of actual plates is, for example, 5 plates or more and 30 plates or less, and preferably 10 plates or more and 25 plates or less. Examples of plates in the plate column include a cross-flow tray and a shower tray. Examples of packing in the packed column include Raschig rings and Lessing rings. Examples of the material of the column and the packing include iron, stainless steel, Hastelloy, borosilicate glass, quartz glass, and a fluororesin (e.g., polytetrafluoroethylene).

The position where the feedstock supply plate is provided in the first distillation column 1 and the second distillation column 2 is not particularly limited, but is preferably three or more theoretical plates below the column top.

Pressures of the first distillation column 1 and the second distillation column 2 are not particularly limited, but are, for example, 0.0 to 0.2 MPa. At this time, temperatures of the column top and the column bottom of the first distillation column 1 and the second distillation column 2 may be appropriately set according to the pressure.

### [Purification Step]

The method for producing the semiconductor cleaning liquid of the present embodiment may further include a purification step of purifying the first bottom liquid to obtain a semiconductor cleaning liquid of the present embodiment.

The purification step preferably includes an azeotropic distillation step in which the first bottom liquid is supplied to a feedstock supply plate of the azeotropic distillation column and is distilled to obtain an azeotropic mixture of isopropyl alcohol and water, a dehydration step in which the azeotropic mixture of isopropyl alcohol and water is dehydrated, and a high-boiling distillation step in which the dehydrated azeotropic mixture is supplied to a feedstock supply plate of the high-boiling distillation column and is distilled to obtain the semiconductor cleaning liquid.

### (Azeotropic Distillation Step)

In the azeotropic distillation step, the first bottom liquid is distilled, and an azeotropic mixture of isopropyl alcohol and water is withdrawn as a distillate from the column bottom of the azeotropic distillation column, and a bottom liquid containing high-boiling point impurities having higher boiling points than isopropyl alcohol is withdrawn from the column bottom of the azeotropic distillation column.

Specifically, since the azeotropic temperature of isopropyl alcohol and water is 80.1° C., the azeotropic mixture of isopropyl alcohol and water is withdrawn from the column top by distilling the first bottom liquid at 80.1° C. On the other hand, at the column bottom of the column, high-boiling point impurities are withdrawn together with water.

In addition, the azeotropic distillation step may be performed according to various conditions described in the low-boiling distillation step.

### (Dehydration Step)

In the dehydration step, the azeotropic mixture of isopropyl alcohol and water obtained in the azeotropic distillation step is dehydrated.

The dehydration method is not particularly limited, and examples thereof include distillation, adsorption, and membrane permeation. When an azeotropic mixture of isopropyl alcohol and water is distilled, water can be removed by adding diethyl ether, benzene, toluene, trichloroethylene, dichloromethane, hexenes, or the like to form a three-component azeotropic composition.

### (High-Boiling Distillation Step)

In the high-boiling distillation step, the azeotropic mixture dehydrated in the dehydration step is distilled, a semiconductor cleaning liquid is withdrawn as a distillate from the column top of the high-boiling distillation column, and the bottom liquid containing high-boiling impurities having higher boiling points than isopropyl alcohol is withdrawn from the column bottom of the high-boiling distillation column.

### (Other Steps)

The semiconductor cleaning liquid obtained in the high-boiling distillation step may be further purified, if necessary, by a method such as adsorption; metal particles, inorganic particles, organic particles and the like may be removed by filter filtration; or metal ions and the like may be removed by an ion exchange resin column.

Although the embodiments of the present invention have been described above, the present invention is not limited to the above-described embodiments, and the above-described embodiments may be appropriately modified within the scope of the gist of the present invention.

### EXAMPLES

Hereinafter, examples of the present invention will be described, but the present invention is not limited to the examples. In the present examples, % and ppm are on a mass basis unless otherwise specified.

### Analysis Example 1

(Water Concentration Measurement of Samples with High Concentration Water)

### Equipment: Karl Fischer Moisture Meter CA-200 (Manufactured by Mitsubishi Chemical Analytical Co., Ltd.)

Samples expected to have a water concentration greater than several percentages were measured after being diluted with IPA. The water concentration in IPA to be used for dilution was measured in advance and confirmed to be 100 ppm or less. Samples expected to have a water concentration of a few percentages or less were analyzed without dilution. When the water content is higher than expected, it takes time to measure the water content, but the measured value is not affected. When measuring a water content of 100 ppm or less, it is preferable to collect 5 g or more of a measurement sample with a Terumo syringe in a glove box having a dew point of −60° C. or lower, and measure it with a Karl Fischer moisture meter. This analysis method is applicable for quantification of water content of 1 ppm or more.

### Analysis Example 2

(Aldehyde and/or Ketone Concentration Measurement)

Aldehyde and/or ketone compounds, particularly those contained in water-containing isopropyl alcohol, were analyzed by the present method. Even when water is not contained, the present method is applicable to the analysis of aldehyde and/or ketone. Each aldehyde and/or ketone compound was converted into a 2,4-dinitrophenylhydrazine (DNPH) derivative, followed by concentration, and quantification of the aldehyde and/or ketone was performed.

100 mg of DNPH and 100 ml of a 2 mol/L hydrochloric acid solution were mixed to prepare a DNPH hydrochloric acid solution. 50 ml of isopropyl alcohol and 1 ml of the DNPH hydrochloric acid solution were mixed, allowed to stand for 1 hour, and concentrated, 50-fold, to 1 ml. The obtained concentrated sample was analyzed by high performance liquid chromatography (HPLC) under the following conditions. As a result of calculating lower limits of quantification using a standard substance, the lower limits of quantification of acetaldehyde, propionaldehyde, crotonaldehyde, and 2-pentanone were 0.1 ppb.

### —Measurement Conditions—

Apparatus: Ultimate 3000 (manufactured by Thermo Fisher Scientific)
Column: Inertsil ODS-2 (manufactured by GL Sciences Inc)
Particle size of column packing: 5 μm
Column diameter: 2.1 mm
Column Length: 250 mm
Flow rate: 0.2 ml/min
Column temperature: 40° C.
Detector: UV (360 nm)
Sample injection amount: 10 μl
Mobile Phase Ratio: 0→14 minutes: acetonitrile/1 mM acetic acid+2 mM ammonium acetate: 48/52 (constant), 14 minutes→25 minutes: acetonitrile/1 mM acetic acid+2 mM ammonium acetate: 48/52→100/0 (gradient), 25 minutes→45 minutes: acetonitrile/1 mM acetic acid+2 mM ammonium acetate: 100/0 (constant)

### Analysis Example 3-1

(Measurement of t-Butyl Alcohol Concentration)

Tertiary-butyl alcohol contained in isopropyl alcohol containing water was measured by a headspace method using GC-MS under the measurement conditions shown below. As a result of calculating the lower limit of quantification using a standard substance, the lower limit of quantification of t-butyl alcohol in a mixture with a water concentration of 95% and an isopropyl alcohol concentration of 5% was 5 ppb.

### —Measurement Conditions—

Apparatus: 7890A/5975C (manufactured by Agilent Technologies)
Analytical column: J&W DB-1 (60 m×0.32 mm, 5 μm)
Column temperature: 35° C. (2 min hold)→temperature rise at 10° C./min→250° C. (6 min hold) Carrier gas: helium
Injection pressure: 20 psi
Linear velocity: 31 cm/sec
Injection inlet temperature: 200° C.
Sample injection method: split method
Split Ratio: 1:5
Injection volume: 1 ml
Headspace heating temperature: 60° C.
Headspace heating time: 20 min
Transfer line temperature: 240° C.
Temperature of ion source and quadrupole: 230° C. and 150° C., respectively
Scan Ions: m/z=25 to 250
SIM monitor ions: 31 and 59

### Analysis Example 3-2

(Measurement of t-Butyl Alcohol Concentration)

Tertiary-butyl alcohol contained in the isopropyl alcohol was measured using GC-MS under the following measurement conditions. As a result of calculating the lower limit of quantification using a standard substance, the lower limit of quantification of t-butyl alcohol was 10 ppb.

### —Measurement Conditions—

Apparatus: 7890A/5975C (manufactured by Agilent Technologies)
Analytical column: J&W DB-1 (60 m×0.32 mm, 5 μm)
Column temperature: 35° C. (2 min hold)→temperature rise at 10° C./min→250° C. (6 min hold)
Carrier gas: helium
Injection pressure: 20 psi
Linear velocity: 31 cm/sec
Inlet temperature: 200° C.
Sample injection method: splitless method
Injection volume: 2 μl
Headspace heating temperature: 60° C.
Headspace heating time: 20 min
Transfer line temperature: 240° C.
Temperature of ion source and quadrupole: 230° C. and 150° C., respectively
Scan ions: m/z=25 to 250
SIM monitor ions: 31 and 59

### Example 1

### (Reaction Step)

Propylene containing propane (40,000 ppm), ethane (20 ppm), 2-butene (5 ppm), isobutene (0.1 ppm or less), pentene (0.1 ppm or less), and hexene (0.1 ppm or less) as impurities was prepared as a feedstock. Further, as water as a feedstock, water was prepared, to which phosphotungstic acid as an acid catalyst was added to adjust pH to 3.0.

Water (density 0.92 kg/L) heated to 110° C. was fed into a reaction column having an internal volume of 10 L at a feed amount of 18.4 kg/h (20 L/h), and propylene was fed at a feed amount of 1.2 kg/h. At this time, propylene and water were allowed to react at a temperature and a pressure in the reaction column of 280° C. and 250 atm, respectively, to obtain a reaction product containing isopropyl alcohol. Next, by setting the temperature and pressure in the propylene recovery column to 140° C. and 18 atm, respectively, propylene dissolved in water contained in the reaction products was recovered as gas to obtain a crude aqueous isopropyl alcohol solution. The recovered propylene was fed into a recovery drum of propylene for reuse as a feedstock. At this time, conversion ratio of propylene was 84.0%, and selectivity of propylene to isopropyl alcohol was 99.2%. A water content of the crude aqueous isopropyl alcohol solution was 95 mass %.

### Low-Boiling Distillation Step (First Distillation Step and Second Distillation Step)

An Oldershaw type low-boiling distillation column (first distillation column) having a plate number of 60 and an Oldershaw-type recovery distillation column (second distillation column) having a plate number of 20 were installed. Here, the first distillation column was a vessel having a column bottom of 2 L, allowing the first bottom liquid to be withdrawn from the column bottom. In the first distillation column, a condenser is provided at the column top (the uppermost plate of the column), a part of the liquid condensed by the condenser is refluxed to the column top, and the remainder is withdrawn as a first distillate. On the other hand, the second distillation column is a vessel having a column bottom of 500 ml, and the second bottom liquid is withdrawn from the column bottom. In addition, the second distillation column is provided with a condenser at the column top, and part of liquid condensed by the condenser is refluxed to the column top, and the remainder is withdrawn as a second distillate.

A crude isopropyl alcohol aqueous solution was supplied at 10 L/h to a feedstock supply plate (8^{th} plate) which was 7 plates below the column top (1^{st} plate) of the first distillation column to distill the crude isopropyl alcohol aqueous solution. At this time, the column top temperature was set to 75 to 85° C., and the column pressure (gauge pressure) was set to 0 to 10 kPa. A reflux amount was set to 2.5 L/h, a reflux ratio was set to about 62.5, and the first distillate was withdrawn from the condenser at 40 ml/h. Further, the first bottom liquid was withdrawn from the column bottom at about 10 L/h so that a liquid amount in the first distillation column was maintained at about 1.5 L.

Further, the first distillate was supplied at 40 ml/h to a feedstock supply plate (5^{th} plate) four plates below the column top of the second distillation column, and the first distillate was distilled. At this time, the column top temperature was set to 50 to 80° C., and the column pressure (gauge pressure) was set to 0 to 10 kPa. The reflux amount was set to 30 mL/h, the reflux ratio was set to about 6, and water was supplied to the column top of the second distillation column at 60 ml/h. The second bottom liquid was withdrawn from the column bottom of the second distillation column at a rate of 95 ml/h and was supplied to the column top of the first distillation column. Further, the second distillate was withdrawn from the condenser of the second distillation column at a rate of 5 ml/h and discharged.

### (Purification Step)

The first bottom liquid was supplied to the feedstock supply plate of the azeotropic distillation column and was distilled to obtain an azeotropic mixture of isopropyl alcohol and water (mass ratio: 87.5:12.5). Next, the azeotropic mixture of isopropyl alcohol and water was dehydrated and then was distilled using a high-boiling distillation column to obtain a semiconductor cleaning liquid.

### (Simulation of Water Content in Liquid Phase in Each Plate of First Distillation Column)

Using process simulation software AspenPlus (manufactured by Aspen Technology Co., Ltd.), the water content in the liquid phase in each plate of the first distillation column was simulated. As a result, the water content in the liquid phase was maintained at 15% by mass or more from the feedstock supply plate (8^{th} plate) to the second plate (see Table 1). At this time, assuming that column efficiency was 708, the water content in the liquid phase was maintained at 15 mass % or more in 4.9, as a theoretical plate number, plates among the plates from the feedstock supply plate to the column top. In order to allow the water content in the liquid phase in three or more, as a theoretical plate number, plates among the plates from the feedstock supply plate to the column top to be 15 mass % or more, it is sufficient for the water content in the liquid phase in a plate (4^{th} plate) which is three plates below the column top of the first distillation column, in other words, four plates above the feedstock supply plate (8^{th} plate) of the first distillation column, to be 15 mass % or more.

### (Measurement of Water Content in Liquid Phase in 4^{th} Plate of the First Distillation Column)

The water content in the liquid phase withdrawn from the 4^{th} plate of the first distillation column was analyzed using analysis example 1 (see Table 1).

### (Measurement of Concentration of Components in First Bottom Liquid)

Contents of components (t-butyl alcohol, water, 2-pentanone, crotonaldehyde, acetaldehyde, propionaldehyde) in the first bottom liquid were analyzed using analysis examples 2 and 4 (see Table 2).

### (Measurement of Contents of Components in Semiconductor Cleaning Liquid)

Contents of components (t-butyl alcohol, 2-pentanone, crotonaldehyde, acetaldehyde, propionaldehyde) in the semiconductor cleaning liquid were analyzed using analysis examples 2 and 4. The semiconductor cleaning liquid had a mass ratio of t-butyl alcohol with respect to isopropyl alcohol of 0.6 ppm (see Table 3). In the semiconductor cleaning liquid, the mass ratio of 2-pentanone with respect to isopropyl alcohol was 20 ppb, and the mass ratio of crotonaldehyde with respect to isopropyl alcohol was 10 ppb.

### Example 2

A semiconductor cleaning liquid was obtained in the same manner as in Example 1 except that, in the Low-Boiling Distillation Step of Crude Isopropyl Alcohol Aqueous Solution, water was supplied to the column top of the second distillation column at a rate of 120 ml/h and the second bottom liquid was withdrawn from the column bottom of the second distillation column at a rate of 155 ml/h and was supplied to the column top of the first distillation column. The semiconductor cleaning liquid had a mass ratio of t-butyl alcohol with respect to isopropyl alcohol of 0.3 ppm. In the semiconductor cleaning liquid, the mass ratio of 2-pentanone with respect to isopropyl alcohol was 20 ppb and the mass ratio of crotonaldehyde with respect to isopropyl alcohol was 8 ppb.

### (Simulation of Water Content in Liquid Phase in Each Plate of First Distillation Column)

Using the process simulation software AspenPlus (manufactured by Aspen Technology Co., Ltd.), the water content in the liquid phase in each plate of the first distillation column was simulated. As a result, the water content in the liquid phase was maintained at 15 mass % or more from the feedstock supply plate (8^{th} plate) to the 1^{st} plate. At this time, assuming that the column efficiency was 70%, the water content in the liquid phase was maintained at 15 mass % or more in 5.6, as a theoretical plate number, plates among the plates from the feedstock supply plate to the column top.

### Example 3

A semiconductor cleaning liquid was obtained in the same manner as in Example 2 except that, in the Low-Boiling Step of Crude Isopropyl Alcohol Aqueous Solution, the second bottom liquid was supplied from the column bottom of the second distillation column to a plate which was three plates above the feedstock supply plate of the first distillation column. The semiconductor cleaning liquid had a mass ratio of t-butyl alcohol with respect to isopropyl alcohol of 0.8 ppm. In the semiconductor cleaning liquid, the mass ratio of 2-pentanone with respect to isopropyl alcohol was 20 ppb and the mass ratio of crotonaldehyde with respect to isopropyl alcohol was 10 ppb.

### (Simulation of Water Content in Liquid Phase in Each Plate of First Distillation Column)

Using the process simulation software AspenPlus (manufactured by Aspen Technology Co., Ltd.), the water content in the liquid phase in each plate of the first distillation column was simulated. As a result, the water content in the liquid phase was maintained at 15 mass % or more from the feedstock supply plate (8^{th} plate) to the 1^{st} plate. At this time, assuming that the column efficiency was 70%, the water content in the liquid phase was maintained at 15 mass % or more in 5.6, as a theoretical plate number, plates among the plates from the feedstock supply plate to the column top.

### Example 4

A semiconductor cleaning liquid was obtained in the same manner as in Example 1 except that, in the Low-Boiling Distillation Step of Crude Isopropyl Alcohol Aqueous Solution, water was supplied to the column top of the second distillation column at a rate of 360 ml/h and the second bottom liquid was withdrawn from the column bottom of the second distillation column at a rate of 390 ml/h and was supplied to the column top of the first distillation column. The semiconductor cleaning liquid had a mass ratio of t-butyl alcohol with respect to isopropyl alcohol of 0.15 ppm. In the semiconductor cleaning liquid, the mass ratio of 2-pentanone with respect to isopropyl alcohol was 20 ppb and the mass ratio of crotonaldehyde with respect to isopropyl alcohol was 7 ppb.

### (Simulation of Water Content in Liquid Phase in Each Plate of First Distillation Column)

Using the process simulation software AspenPlus (manufactured by Aspen Technology Co., Ltd.), the water content in the liquid phase in each plate of the first distillation column was simulated. As a result, the water content in the liquid phase was maintained at 15 mass % or more from the feedstock supply plate (8^{th} plate) to the 4^{th} plate. At this time, assuming that the column efficiency was 70%, the water content in the liquid phase was maintained at 15 mass % or more in 3.5, as a theoretical plate number, plates among the plates from the feedstock supply plate to the column top.

### Comparative Example 1

A semiconductor cleaning liquid was obtained in the same manner as in Example 1 except that, in the Low-Boiling Distillation Step of Crude Isopropyl Alcohol Aqueous Solution, water was not supplied to the column top of the second distillation column and the second bottom liquid was withdrawn from the column bottom of the second distillation column at a rate of 35 ml/h and was supplied to the column top of the first distillation column. The semiconductor cleaning liquid had a mass ratio of t-butyl alcohol with respect to isopropyl alcohol of 2.2 ppm. In the semiconductor cleaning liquid, the mass ratio of 2-pentanone with respect to isopropyl alcohol was 20 ppb and the mass ratio of crotonaldehyde with respect to isopropyl alcohol was 10 ppb.

In addition, as a result of simulation of the water content in the liquid phase in each plate of the first distillation column, the water content in the liquid phase was maintained at 15% by mass or more from the feedstock supply plate (the 8^{th} plate) to the 5^{th} plate. At this time, assuming that the column efficiency was 708, the water content in the liquid phase was maintained at 15 mass % or more in 2.8, as a theoretical plate number, plates among the plates from the feedstock supply plate to the column top.

### Comparative Example 2

A semiconductor cleaning liquid was obtained in the same manner as in Example 2 except that, in the Low-Boiling Distillation Step of Crude Isopropyl Alcohol Aqueous Solution, the second bottom liquid was supplied from the column bottom of the second distillation column to a plate which was one plate above the feedstock supply plate of the first distillation column. The semiconductor cleaning liquid had a mass ratio of t-butyl alcohol with respect to isopropyl alcohol of 1.5 ppm. In the semiconductor cleaning liquid, the mass ratio of 2-pentanone with respect to isopropyl alcohol was 20 ppb and the mass ratio of crotonaldehyde with respect to isopropyl alcohol was 10 ppb.

### (Simulation of Water Content in Liquid Phase in Each Plate of First Distillation Column)

Using the process simulation software AspenPlus (manufactured by Aspen Technology Co., Ltd.), the water content in the liquid phase in each plate of the first distillation column was simulated. As a result, the water content in the liquid phase was maintained at 15 mass % or more from the feedstock supply plate (8^{th} plate) to the 5^{th} plate. At this time, assuming that the column efficiency was 70%, the water content in the liquid phase was maintained at 15 mass % or more in 2.8, as a theoretical plate number, plates among the plates from the feedstock supply plate to the column top.

### Comparative Example 3

A semiconductor cleaning liquid was obtained in the same manner as in Example 3 except that, in the Low-Boiling Distillation Step of Crude Isopropyl Alcohol Aqueous Solution, water was supplied to the column top of the second distillation column at a rate of 40 ml/h and the second bottom liquid was withdrawn from the column bottom of the second distillation column at a rate of 75 ml/h and was supplied to the column top of the first distillation column. The semiconductor cleaning liquid had a mass ratio of t-butyl alcohol with respect to isopropyl alcohol of 2.0 ppm. In the semiconductor cleaning liquid, the mass ratio of 2-pentanone with respect to isopropyl alcohol was 20 ppb and the mass ratio of crotonaldehyde with respect to isopropyl alcohol was 10 ppb.

### (Simulation of Water Content in Liquid Phase in Each Plate of First Distillation Column)

Using the process simulation software AspenPlus (manufactured by Aspen Technology Co., Ltd.), the water content in the liquid phase in each plate of the first distillation column was simulated. As a result, the water content in the liquid phase was maintained at 15 mass % or more from the feedstock supply plate (8^{th} plate) to the 5^{th} plate. At this time, assuming that the column efficiency was 70%, the water content in the liquid phase was maintained at 15 mass % or more in 2.8, as a theoretical plate number, plates among the plates from the feedstock supply plate to the column top.

Table 1 shows simulation results of the water content in the liquid phase in each plate of the first distillation column, the theoretical plate number of plates where the water content in the liquid phase was 15% by mass or more, and the measurement results of the water content in the liquid phase in the 4^{th} plate.

TABLE 1

| | | | | | | | | | | | Number of theoretical | |
|---|---|---|---|---|---|---|---|---|---|---|---|---|
| | Water content in liquid phase in each plate (%) | | | | | | | | | | plates where water | Water content in liquid |
| | (simulation result) | | | | | | | | | | content in liquid phase | phase in 4th plate (%) |
| | 10 | 9 | 8 | 7 | 6 | 5 | 4 | 3 | 2 | 1 | is 15% or more | (measurement result) |
| Example 1 | 84.2 | 84.1 | 30.1 | 23.4 | 20.5 | 18.9 | 18.1 | 17.6 | 17.2 | 14.6 | 4.2 | 18.21 |
| Example 2 | 84.0 | 84.0 | 32.3 | 25.5 | 22.8 | 21.4 | 20.7 | 20.3 | 20.1 | 15.7 | 4.9 | 20.06 |
| Example 3 | 84.5 | 84.5 | 32.9 | 25.9 | 23.1 | 17.6 | 15.8 | 14.7 | 13.9 | 13.1 | 3.5 | 15.58 |
| Example 4 | 83.8 | 83.8 | 38.6 | 32.5 | 30.3 | 29.4 | 29.1 | 28.8 | 28.7 | 17.9 | 4.9 | 29.45 |
| Comparative | 84.3 | 84.3 | 28.6 | 21.2 | 17.9 | 16.1 | 14.9 | 14.2 | 13.7 | 13.0 | 2.8 | 14.75 |
| Example 1 | | | | | | | | | | | | |
| Comparative | 84.4 | 84.4 | 33. | 21.8 | 18.1 | 16.1 | 14.9 | 14.1 | 13.5 | 12.9 | 2.8 | 14.66 |
| Example 2 | | | | | | | | | | | | |
| Comparative | 84.4 | 84.4 | 23.9 | 21.6 | 18.3 | 16.1 | 14.9 | 14.1 | 13.5 | 12.9 | 2.8 | 14.80 |
| Example 3 | | | | | | | | | | | | |

Table 2 shows contents of components in the first bottom liquid. Analysis examples 1, 2, and 3-1 were used.

TABLE 2

| | Contents of components in first bottom liquid (ppm) | | | | | |
|---|---|---|---|---|---|---|
| | t-butyl | | 2- | | | |
| | alcohol | Water | pentanone | Crotonaldehyde | Acetaldehyde | Propionaldehyde |
| Example 1 | 0.03 | 940000 | 0.001 | 0.0005 | 0.019 | 0.02 |
| Example 2 | 0.015 | 940000 | 0.001 | 0.0004 | 0.07 | 0.02 |
| Example 3 | 0.04 | 940000 | 0.001 | 0.0005 | 0.2 | 0.02 |
| Example 4 | 0.007 | 940000 | 0.001 | 0.0004 | 0.05 | 0.01 |
| Comparative | 0.11 | 940000 | 0.001 | 0.0005 | 0.033 | 0.03 |
| Example 1 | | | | | | |
| Comparative | 0.064 | 940000 | 0.001 | 0.0005 | 0.025 | 0.03 |
| Example 2 | | | | | | |
| Comparative | 0.1 | 940000 | 0.001 | 0.0005 | 0.03 | 0.03 |
| Example 3 | | | | | | |

Table 3 shows mass ratios of components with respect to isopropyl alcohol. Analysis examples 1, 2, and 3-2 were used.

TABLE 3

| | Mass ratio with respect to isopropyl alcohol (ppm) | | | | | |
|---|---|---|---|---|---|---|
| | t- | | 2- | | | |
| | butyl | Wat- | penta- | Croton- | Acetal- | Propion- |
| | alcohol | er | none | aldehyde | dehyde | aldehyde |
| Example 1 | 0.6 | 10 | 0.02 | 0.01 | 0.01 | 0.001 |
| Example 2 | 0.3 | 10 | 0.02 | 0.008 | 0.007 | 0.001 |
| Example 3 | 0.8 | 10 | 0.02 | 0.01 | 0.02 | 0.001 |
| Example 4 | 0.15 | 10 | 0.02 | 0.007 | 0.005 | 0.001 |
| Comparative | 2.2 | 10 | 0.03 | 0.01 | 0.02 | 0.001 |
| Example 1 | | | | | | |
| Comparative | 1.3 | 10 | 0.02 | 0.01 | 0.02 | 0.001 |
| Example 2 | | | | | | |
| Comparative | 2 | 10 | 0.03 | 0.01 | 0.02 | 0.001 |
| Example 3 | | | | | | |

From Table 3, it can be seen that in the semiconductor cleaning liquids of Examples 1 to 4, mass ratios of t-butyl alcohol with respect to isopropyl alcohol were 1 ppm or less. Contrary to this, in the semiconductor cleaning liquids of comparative examples 1 to 3, the water contents in the liquid phases of the first distillation columns were maintained at 15% by mass or more in 2.8, as a theoretical plate number, plates from the feedstock supply plate to the column top, and the mass ratio of t-butyl alcohol with respect to isopropyl alcohol exceeded 1 ppm.

### EXPLANATION OF REFERENCE NUMERALS

1 low-boiling distillation column (first distillation column)
2 recovery distillation column (second distillation column)

1. A semiconductor cleaning liquid comprising isopropyl alcohol,
a mass ratio of t-butyl alcohol with respect to isopropyl alcohol being 1 ppm or less.

2. The semiconductor cleaning liquid according to claim 1,
wherein a mass ratio of 2-pentanone with respect to isopropyl alcohol is 1 ppb or more and 50 ppb or less, and
a mass ratio of croton aldehyde with respect to isopropyl alcohol is 0.5 ppb or more and 10 ppb or less.

3. The semiconductor cleaning liquid according to claim 1,
wherein the isopropyl alcohol is produced by a direct hydration method.

4. A method for producing a semiconductor cleaning liquid,
the method comprising a first distillation step of supplying a crude isopropyl alcohol aqueous solution comprising t-butyl alcohol as an impurity to a feedstock supply plate of a first distillation column, withdrawing a first distillate comprising a low-boiling point impurity having a lower boiling point than isopropyl alcohol from a column top of the first distillation column, and withdrawing a first bottom liquid from a column bottom of the first distillation column,
wherein a fluid comprising water is supplied from outside the first distillation column to a predetermined plate which is two or more theoretical plates above the feedstock supply plate of the first distillation column so that a water content in a liquid phase is 15 mass % or more in three or more, as a theoretical plate number, plates among plates of the first distillation column from the feedstock supply plate to the column top.

5. The method for producing a semiconductor cleaning liquid according to claim 4,
wherein the method further comprises a second distillation step of supplying the first distillate to a feedstock supply plate of a second distillation column, withdrawing a second distillate comprising the low-boiling point impurity from the column top of the second distillation column, and withdrawing a second bottom liquid from the column bottom of the second distillation column, and
in the second distillation step, water is supplied to a predetermined plate of the second distillation column from outside the second distillation column, and
in the first distillation step, the second bottom liquid is supplied as the liquid comprising water.

6. The method for producing a semiconductor cleaning liquid according to claim 5, wherein the predetermined plate of the second distillation column is a plate between the feedstock supply plate of the second distillation column and the column top thereof.

7. The method for producing a semiconductor cleaning liquid according to claim 4, further comprising a reaction step of obtaining the crude isopropyl alcohol aqueous solution by direct hydration of propylene.
